(19) United States
(12) Patent Application Publication
(10) Pub. No.: US 2009/0017415 A1
(43) Pub. Date: Jan. 15, 2009

(54) Heated dental mirror

(75) Inventor: Steven Louis Cornelius, Taft, TN (US)

Correspondence Address: STEVEN L. CORNELIUS, 9 BLANCHE CIRCLE, TAFT, TN, 38488, US

(73) Assignee: Steven Cornelius

(21) Appl. No.: 11/825,608

(22) Filed: Jul. 9, 2007

(51) Int. Cl. A61B 1/24

(52) U.S. Cl. 433/30

(57) ABSTRACT

Sanitary dental mirror made of stainless steel or high-grade aluminum heated dental mirror powered by disposable AAA batteries and sealed with O-Rings. The heated mirror surface doesn't require constant rubbing on smocks and/or napkins to remove moisture vapor that collects due to the temperature differential between the metal mirror and the inside of the patient's mouth. This makes the instrument much more sanitary than mirrors currently in use.

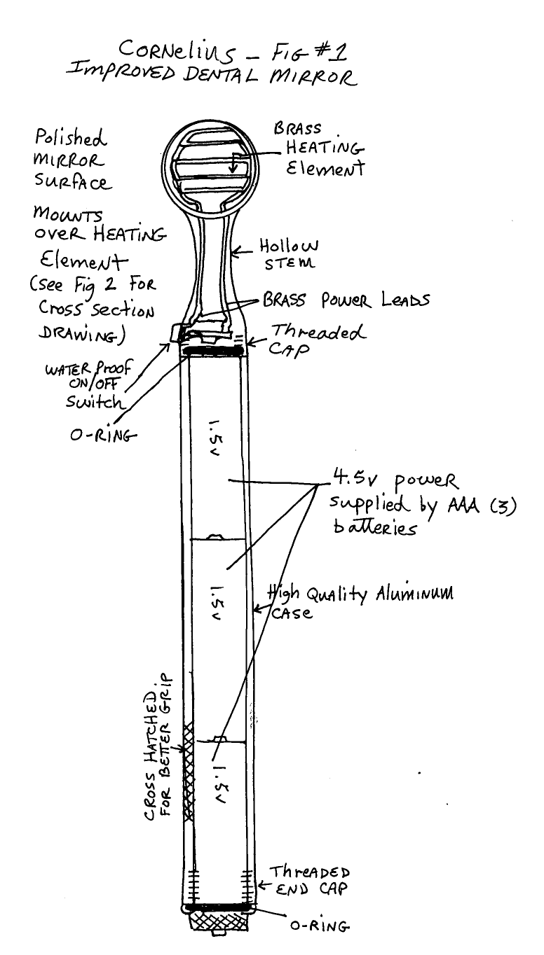

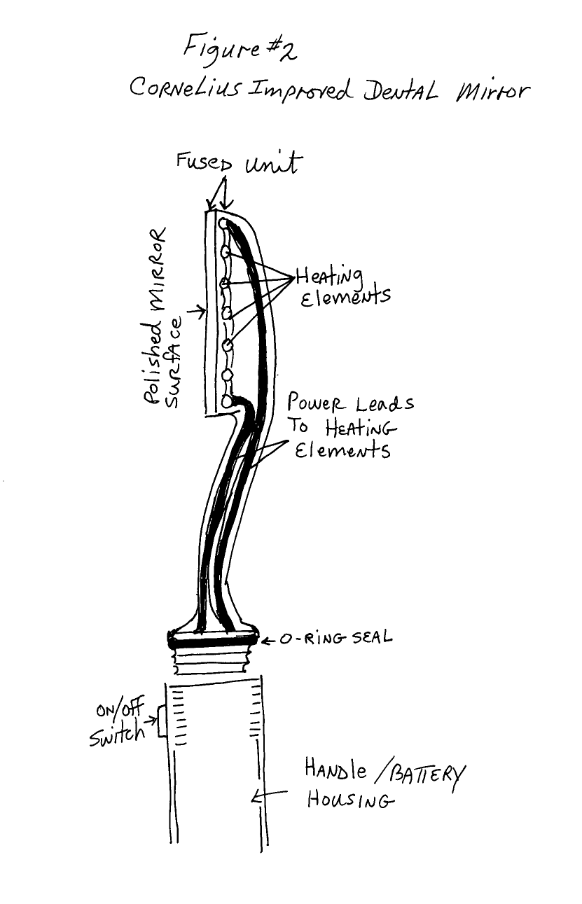

Stainless steel or high-grade hollow aluminum mirror body with highly polished metal or glass reflecting surface. The hollow handle houses three 1.5 volt AAA batteries that supply current to a heating element located behind the reflective mirror surface. The heating element heats the mirror surface to the point that it will not fog up from moisture vapor and heat when inserted into a patient's mouth. This is a more sanitary way of keeping the examination instrument clean and fog free. Typically, when the mirror surface fogs up, the dentist will use a cloth or his smock to rub away the moisture so that he/she can see clearly. A heated mirror would eliminate this unsanitary step as there wouldn't be sufficient temperature difference to cause the moisture to condense into fog on the mirror surface.

### CROSS-REFERENCE TO RELATED APPLICATION

None

### STATEMENT REGARDING FEDERALLY SPONSORED RESEARCH OR DEVELOPMENT

No Federal research or development funds were used in the development of this concept/product.

### REFERENCE TO SEQUENCE LISTING, A TABLE OR A COMPUTER PROGRAM LISTING COMPACT DISC APPENDIX

None

### BACKGROUND OF THE INVENTION

During many visits to the dentist, I observed the dentist constantly removing the mirror from my mouth and rubbing it on his smock to remove moisture vapor that had condensed on the surface from my breath. It occurred to me that this could hardly be a sanitary way to solve the problem at hand. Further, the dentist repeated this action many times during a one hour visit. To pass the time while all the drilling and grinding was going on in my mouth, I thought about ways to eliminate this annoying and unhealthy practice concerning the examination mirror. I quickly realized that if a mirror was heated to approximately the same temperature as the inside of the patient's mouth, or perhaps a degree or two warmer, then the moisture from exhaled breath would not condense and obscure the dentist's view as he worked. This patent submission represents my proposed solution.

### BRIEF SUMMARY OF THE INVENTION

Three piece device made of stainless steel or high grade aluminum, milled into the necessary mirror shape (long slender handle and oval or round reflective head, tilted 30-45 degrees for visibility. The handle will be hollow and will house three batteries. The reflective head will house the heating coil and on/off pressure switch. Each section will be joined together using o-rings to make the assembly water proof. This will allow the device to be autoclaved as one unit or broken down into three separate pieces for sterilization.

### BRIEF DESCRIPTION OF THE SEVERAL VIEWS OF THE DRAWINGS

Drawing #1 depicts a cross section of the entire device, showing the battery housing, contact plates and on/off switch, as well as the reflective head and waterproof end cap.

Drawing #2 illustrates the reflective head with heating coil located just beneath the reflective mirror surface.

### DETAILED DESCRIPTION OF THE INVENTION

Constructed from stainless steel or high-grade hollow aluminum mirror body with highly polished metal or glass reflecting surface. The hollow handle houses three 1.5 volt AAA batteries that supply current to a heating element located behind the reflective mirror surface. The heating element heats the mirror surface to the point that it will not fog up from moisture vapor and heat when inserted into a patient's mouth. This is a more sanitary way of keeping the examination instrument clean and fog free. Typically, when the mirror surface fogs up, the dentist will use a cloth or his smock to rub away the moisture so that he/she can see clearly. A heated mirror would eliminate this unsanitary step as there wouldn't be sufficient temperature difference to cause the moisture to condense into fog on the mirror surface.
1) Mirror handle is milled with cross-hatches to ensure a non-slip, grippable surface;
2) Uses three AAA (1.5 volt) batteries to supply current to the heating element;
3) The entire mirror is capable of being broken into three discreet pieces for cleaning and repair;
4) A pressure switch will activate the heating element. This conserves power and prevents premature heating coil burn-out.
5) The mirror surface may be highly polished steel (cobalt) or glass and tilted from 30-50 degrees to facilitate examination of any tooth/gum area.

1. The improved, heated dental examination mirror is unique in that it uses commonly available batteries to heat an imbedded metal heating element located just under the reflective mirror surface (see illustration/drawing #2). Currently, dental mirrors are constructed of high-grade aluminum or stainless steel, but are approximately 25 degrees cooler than the inside of the patient's mouth. This results in water condensing on the metal/glass surface as the patient exhales. The improved, heated mirror eliminates condensation by heating the reflective surface to 100-105 degrees F. This eliminates the temperature differential and prevents water vapor condensation during dental exams. Key design and manufacturing characteristics are:
I. Incorporates three 1.5 volt disposable batteries—the batteries are housed in the handle of the mirror;
II. Uses heat-resistant rubber O-Rings to seal the battery and mirror areas of the device—allowing the instrument to be sterilized in an autoclave;
III. Candidate construction material is stainless steel or aircraft quality aluminum alloy;
IV. A spring loaded pressure switch located near the dentist's index finger permits activating the heating element “on demand” conserving battery power;
V. Size (6.5 inches in length) and weight (six ounces) of the heated examination mirror are within ½ inch in length and 2 ounces in weight of the majority of dental mirrors currently in use.
